SEQUENCE LISTING (21 sequences)

SEQ ID NO: 1 - protein, 479 aa, Escherichia coli
MSIVVKNNIHWVGQRDWEVRDFHGTEYKTLRGSSYNSYLIREEKNVLIDTVDHKFSREFVQNLRNEIDLADIDYIVINHAEEDHAGALTELMAQIPDTPIYCTANAIDSINGHHHHPEWNFNVVKTGDTLDIGNGKQLIFVETPMLHWPDSMMTYLTGDAVLFSNDAFGQHYCDEHLFNDEVDQTELFEQCQRYYANILTPFSRLVTPKITEILGFNLPVDMIATSHGVVWRDNPTQIVELYLKWAADYQEDRITIFYDTMSNNTRMMADAIAQGIAETDPRVAVKIFNVARSDKNEILTNVFRSKGVLVGTSTMNNVMMPKIAGLVEEMTGLRFRNKRASAFGSHGWSGGAVDRLSTRLQDAGFEMSLSLKAKWRPDQDALKLCREHGREIARQWALAPLPQSTVNTVVKEETSATTTADLGPRMQCSVCQWIYDPAKGEPMQDVAPGTPWSEVPDNFLCPECSLGKDVFEELASEAK

SEQ ID NO: 2 - protein, 377 aa, Escherichia coli
MSNGIVIIGSGFAARQLVKNIRKQDATIPLTLIAADSMDEYNKPDLSHVISQGQRADDLTRQTAGEFAEQFNLHLFPQTWVTDIDAEARVVKSQNNQWQYDKLVLATGASAFVPPVPGRELMLTLNSQQEYRACETQLRDARRVLIVGGGLIGSELAMDFCRAGKAVTLIDNAASILASLMPPEVSSRLQHRLTEMGVHLLLKSQLQGLEKTDSGIQATLDRQRNIEVDAVIAATGLRPETALARRAGLTINRGVCVDSYLQTSNTDIYALGDCAEINGQVLPFLQPIQLSAMVLAKNLLGNNTPLKLPAMLVKIKTPELPLHLAGETQRQDLRWQINTERQGMVARGVDDADQLRAFVVSEDRMKEAFGLLKTLPM

SEQ ID NO: 3 - protein, 462 aa, Paracoccus versutus DSM 582
MRYHSQRIAYAYFLVAMALFAVQVIFGLIMGWIYVSPNFLSELLPFNIARMLHTNSLVVWLLLGFFGATYYILPEEAEREIHSPMLAYIQLGIFVLGTLGVVVTYLFDLFHGHWLLGKEGREFLEQPKWVKVGIAVAAVIFMYNVSMTALKGRRTAVTNVLLMGLWGLVLLWLFAFYNPANLVLDKQYWWWVIHLWVEGVWELIMAAILAFLMLKLTGVDREVVEKWLYVIVATALFSGILGTGHHYYWIGLPAYWQWIGSIFSSFEIVPFFAMMSFAFVMVWKGRRDHPNKAALVWSLGCTVLAFFGAGVWGFLHTLHGVNYYTHGTQITAAHGHLAFYGAYVCLVLALVTYCMPLMKNRDPYNQVLNMASFWLMSSGMVFMTVTLTFAGTVQTHLQRVEGGFFMDVQDGLALFYWMRFGSGVAVVLGALLFIYAVLFPRREVVTAGPVQAHKDGHLEAAE

SEQ ID NO: 4 - protein, 150 aa, Paracoccus versutus DSM 582
MSEIMTKNMARNVFYGGSIFFILIFGALTVHSHIYARTKAVDESQLTPSVAEGKHIWERNACIDCHTILGEGAYFAPELGNVMKRWGVQDDPESAFETLKGWMESMPTGIEGRRQMPRFDLTDEEFRALSDFLLWTGTINTQNWPPNDAG

SEQ ID NO: 5 - DNA, 1440 bp, Escherichia coli
atgtctattgtggtgaaaaataacattcattgggttggtcaacgtgactgggaagtgcgtgattttcacggcacggaatataaaacgctgcgcggcagcagctacaatagctacctcatccgcgaagaaaaaaacgtgctgatcgacaccgtcgaccataaattcagccgcgaatttgtgcagaacctgcgtaatgaaatcgatctggcggatatcgattacatcgtgattaaccatgcagaagaggaccacgctggggcgctgaccgaactgatggcacaaattcccgatacgccgatctactgtacagccaacgctatcgactcgataaatggtcatcaccatcatccggagtggaattttaatgtggtgaaaactggcgacacgctggatatcggcaacggcaaacagctcatttttgtcgaaacaccaatgctgcactggccggacagcatgatgacttacctgacaggcgacgcggtgctgttcagtaacgatgctttcggtcaacactactgcgacgagcatctgttcaacgatgaagtggatcagacggagcttttcgagcagtgccagcgttactacgccaatatcctgacgccgttcagccgcctggtaacaccgaaaattaccgagatcctgggctttaacttaccagtcgatatgatagccacttcccacggcgtggtatggcgcgataacccgacgcaaattgtcgagctgtacctgaaatgggcggctgattatcaggaagacagaatcaccattttctacgacaccatgtcgaataacacccgcatgatggctgacgctatcgcccaggggattgcggaaaccgacccacgcgtggcggtgaaaattttcaacgtcgcccgaagcgataaaaacgaaatcctgactaatgtcttccgctcaaaaggcgtgctggtcggcacttcgacgatgaataacgtgatgatgccgaaaatcgccgggctggtggaggagatgactggtttacgcttccgtaacaaacgcgccagtgctttcggctctcacggctggagcggcggtgcggtggatcgtctttccacgcgcctgcaggatgcgggtttcgaaatgtcgcttagcctgaaagcgaaatggcgaccagaccaggacgctctgaagttatgccgtgaacacggtcgcgaaatcgcccgtcagtgggcgctcgcgccgctgccgcagagcacggtgaatacggtagttaaagaagaaacctctgccaccacgacggctgacctcggcccacggatgcagtgcagcgtctgccagtggatttacgatccggcaaaaggcgagccaatgcaggacgttgcgccaggaacgccgtggagtgaagtcccggataacttcctctgcccggaatgctccctcggcaaagacgtctttgaagaactggcatcggaggcaaaatga

SEQ ID NO: 6 - DNA, 1134 bp, Escherichia coli
atgagtaacggcattgtgatcatcggttcgggcttcgccgcccgccaactggtgaaaaatattcgcaaacaggacgccactattccattaaccctgattgccgccgacagcatggatgagtacaacaaacctgacctcagccatgttatcagtcaggggcaacgtgccgatgaccttacccgccagacggcgggtgaatttgccgagcagtttaatctgcacctgtttccacaaacctgggtgacggatatcgatgccgaagcccgtgtggtgaaaagccagaataatcagtggcaatacgacaagctagtactggcaaccggtgccagtgcctttgtcccgcctgtgcctgggcgtgagttaatgctgacgttaaatagtcagcaagagtatcgcgcctgtgaaacgcaactgcgggatgcccgacgcgtgttgattgttggcggtggtttgattggtagcgaactggcgatggatttttgtcgtgcaggcaaagcggtcacgctaatcgacaacgctgccagtattctggcgtcgttaatgccaccggaagtaagcagccgcttgcagcatcggttgacggagatgggcgttcatctgctgttgaaatctcagttacaggggctggaaaaaacggattctggcattcaggcaacgctggaccgccagcgcaatatcgaagtggatgcggtaattgccgccaccggactgcgcccggaaaccgccctggcacgacgcgccgggctgacgattaatcgcggcgtttgcgtcgatagttatctgcaaaccagtaataccgatatttacgcgctgggcgattgcgcggaaattaacggtcaggtattgccgttcctccagccgattcaacttagcgcgatggtgctggcaaaaaatcttctcggcaataacacgccgctgaaactcccggcgatgctggtgaaaatcaaaacgccggaattaccgctgcatctggcaggcgaaacccagcgtcaggatttacgctggcaaattaataccgaacgccagggaatggtggcgcgcggcgttgacgatgctgaccagcttcgcgcctttgtggtcagtgaggatcggatgaaagaggcatttggattgttgaaaacattgccgatgtag

SEQ ID NO: 7 - DNA, 1389 bp, Paracoccus versutus DSM 582
atgagataccattcgcaacgcatcgcctatgcctatttcctggtggccatggcgcttttcgccgttcaggtgattttcggcctgatcatgggctggatctatgtcagcccgaatttcctgtccgaattgctgccctttaatatcgcgcggatgctgcataccaacagcctggtcgtctggctgctcttgggctttttcggcgcgacctattacatcctgcccgaagaggccgagcgcgaaatccattcgccgatgctggcctatatccagctgggcattttcgtgctgggcacgctgggcgtggtcgtgacctatctgttcgaccttttccatggccattggcttttgggcaaggaggggcgcgagttcctggaacaaccgaaatgggtcaaggtcggcatcgccgtggccgcggtgatcttcatgtacaacgtcagcatgaccgcgctgaagggccggcggacggcggtcacgaacgtgctgctgatgggcctgtgggggctggtgctgctgtggctctttgccttctacaacccggcgaacctggtcctcgacaagcaatactggtggtgggtgatccacctgtgggtcgagggcgtgtgggagctgatcatggccgccatcctcgccttcctgatgctcaagctgaccggcgtggaccgcgaggtggtcgagaaatggctttacgtcatcgtcgccacggcgctgttctcgggcatcctgggcaccgggcaccactactactggatcggcctgccggcctattggcagtggatcggctcgatcttctcgagcttcgagatcgtgcccttcttcgcgatgatgtcctttgccttcgtcatggtctggaagggccggcgcgaccacccgaacaaggcggcgctggtctggagcctgggctgcaccgtgctggccttcttcggggccggggtctggggcttcctgcacacgctgcacggggtgaactactatacccacggcacgcagatcaccgccgcgcatggccacctggccttctatggcgcctatgtctgcctggtgctggcgctggtgacctattgcatgccgctgatgaagaaccgcgacccctacaaccaggtgctgaacatggcctcgttctggctgatgtcctcgggcatggtgttcatgacggtgacgctgaccttcgccggcacggtgcagacccatctgcagcgggtcgagggcgggttcttcatggacgtgcaggacgggctggcgctgttctactggatgcgcttcggctcgggcgtcgcggtggtgctgggcgcgctgctcttcatctatgcggtgctgttcccgcgccgcgaggtggtcacagccggcccggtgcaggcgcacaaggacggccacctggaggctgcggagtaa

SEQ ID NO: 8 - DNA, 453 bp, Paracoccus versutus DSM 582
atgagcgaaatcatgaccaagaacatggcccggaacgtcttctacggcgggtccatattcttcatcctgatctttggcgcgttaaccgtgcatagccatatctatgcccgcaccaaggcagtggatgaaagccagctcaccccctcggtcgccgagggcaagcatatctgggaacgcaacgcctgcattgactgccataccatcctgggcgagggcgcctatttcgcccccgagcttggcaatgtgatgaaacgctggggcgtgcaggatgatcccgaatccgccttcgagacgctgaagggctggatggaatccatgcccaccggaatcgagggtcgccgccagatgccccgcttcgatctgacggacgaggaattccgggcgctgtcggatttcctgctgtggaccggcacgatcaacacccagaactggccaccgaacgacgcgggctga

SEQ ID NO: 9 - DNA, 70 bp, Artificial
  primer
gcaattagcaagacatctttttagaacacgctgaataaattgaggttgctgtgtaggctggagctgcttc

SEQ ID NO: 10 - DNA, 70 bp, Artificial
  primer
ggataagacgcgccagcgtcgcatccgacattttaggcacagtagcccaccatatgaatatcctccttag

SEQ ID NO: 11 - DNA, 20 bp, Artificial
  primer
tcatctttgcctcactgtca

SEQ ID NO: 12 - DNA, 20 bp, Artificial
  primer
gaagtccagaacggataaaa

SEQ ID NO: 13 - DNA, 38 bp, Artificial
  primer
taaagaggagaaattaaccatgtctattgtggtgaaaa

SEQ ID NO: 14 - DNA, 39 bp, Artificial
  primer
ccaagctcagctaattaagcttacatcggcaatgttttc

SEQ ID NO: 15 - DNA, 38 bp, Artificial
  primer
ttttcaccacaatagacatggttaatttctcctcttta

SEQ ID NO: 16 - DNA, 39 bp, Artificial
  primer
gaaaacattgccgatgtaagcttaattagctgagcttgg

SEQ ID NO: 17 - DNA, 39 bp, Artificial
  primer
taaagaggagaaattaaccatgagcgaaatcatgaccaa

SEQ ID NO: 18 - DNA, 39 bp, Artificial
  primer
ccaagctcagctaattaagcttactccgcagcctccagg

SEQ ID NO: 19 - DNA, 39 bp, Artificial
  primer
ttggtcatgatttcgctcatggttaatttctcctcttta

SEQ ID NO: 20 - DNA, 39 bp, Artificial
  primer
cctggaggctgcggagtaagcttaattagctgagcttgg

SEQ ID NO: 21 - DNA, 1854 bp, Artificial
  A nucleotide sequence for norCB gene
atgagcgaaatcatgaccaagaacatggcccggaacgtcttctacggcgggtccatattcttcatcctgatctttggcgcgttaaccgtgcatagccatatctatgcccgcaccaaggcagtggatgaaagccagctcaccccctcggtcgccgagggcaagcatatctgggaacgcaacgcctgcattgactgccataccatcctgggcgagggcgcctatttcgcccccgagcttggcaatgtgatgaaacgctggggcgtgcaggatgatcccgaatccgccttcgagacgctgaagggctggatggaatccatgcccaccggaatcgagggtcgccgccagatgccccgcttcgatctgacggacgaggaattccgggcgctgtcggatttcctgctgtggaccggcacgatcaacacccagaactggccaccgaacgacgcgggctgagggggaaaaccgatgagataccattcgcaacgcatcgcctatgcctatttcctggtggccatggcgcttttcgccgttcaggtgattttcggcctgatcatgggctggatctatgtcagcccgaatttcctgtccgaattgctgccctttaatatcgcgcggatgctgcataccaacagcctggtcgtctggctgctcttgggctttttcggcgcgacctattacatcctgcccgaagaggccgagcgcgaaatccattcgccgatgctggcctatatccagctgggcattttcgtgctgggcacgctgggcgtggtcgtgacctatctgttcgaccttttccatggccattggcttttgggcaaggaggggcgcgagttcctggaacaaccgaaatgggtcaaggtcggcatcgccgtggccgcggtgatcttcatgtacaacgtcagcatgaccgcgctgaagggccggcggacggcggtcacgaacgtgctgctgatgggcctgtgggggctggtgctgctgtggctctttgccttctacaacccggcgaacctggtcctcgacaagcaatactggtggtgggtgatccacctgtgggtcgagggcgtgtgggagctgatcatggccgccatcctcgccttcctgatgctcaagctgaccggcgtggaccgcgaggtggtcgagaaatggctttacgtcatcgtcgccacggcgctgttctcgggcatcctgggcaccgggcaccactactactggatcggcctgccggcctattggcagtggatcggctcgatcttctcgagcttcgagatcgtgcccttcttcgcgatgatgtcctttgccttcgtcatggtctggaagggccggcgcgaccacccgaacaaggcggcgctggtctggagcctgggctgcaccgtgctggccttcttcggggccggggtctggggcttcctgcacacgctgcacggggtgaactactatacccacggcacgcagatcaccgccgcgcatggccacctggccttctatggcgcctatgtctgcctggtgctggcgctggtgacctattgcatgccgctgatgaagaaccgcgacccctacaaccaggtgctgaacatggcctcgttctggctgatgtcctcgggcatggtgttcatgacggtgacgctgaccttcgccggcacggtgcagacccatctgcagcgggtcgagggcgggttcttcatggacgtgcaggacgggctggcgctgttctactggatgcgcttcggctcgggcgtcgcggtggtgctgggcgcgctgctcttcatctatgcggtgctgttcccgcgccgcgaggtggtcacagccggcccggtgcaggcgcacaaggacggccacctggaggctgcggagtaa

(19) United States
(12) Patent Application Publication
(10) Pub. No.: US 2023/0183755 A1
(43) Pub. Date: Jun. 15, 2023

(54) RECOMBINANT MICROORGANISM HAVING ENHANCED ABILITY TO REMOVE NITRIC OXIDE AND USE THEREOF

(71) Applicant: Samsung Electronics Co., Ltd., Suwon-si (KR)

(72) Inventors: Yu Kyung Jung, Hwaseong-si (KR); Jae-Young Kim, Suwon-si (KR); Seung Hoon Song, Suwon-si (KR); Woo Yong Shim, Hwaseong-si (KR)

(21) Appl. No.: 17/828,332

(22) Filed: May 31, 2022

(30) Foreign Application Priority Data
Dec. 13, 2021 (KR) 10-2021-0178036

(51) Int. Cl. C12P 1/04; B01D 53/56; C12N 1/20; C12N 9/06; C12N 15/70

(52) U.S. Cl. CPC C12P 1/04; B01D 53/56; C12N 1/20; C12N 9/0044; C12N 15/70; C12Y 107/02005

(57) ABSTRACT

A recombinant microorganism including a genetic modification that increases expression of a gene encoding a ferric enterobactin transporter-associated protein or a gene encoding a TonB-dependent transporter-associated protein, or a genetic modification that decreases expression of a fur gene encoding a ferric uptake regulator (Fur) protein, wherein the recombinant microorganism removes greater amounts of nitric oxide from a sample comprising nitric oxide than a same microorganism without the genetic modification

### CROSS-REFERENCE TO RELATED APPLICATION

This application is based on and claims priority to Korean Patent Application No. 10-2021-0178036, filed on Dec. 13, 2021, in the Korean Intellectual Property Office, and all the benefits accruing therefrom under 35 U.S.C. §119, the content of which in its entirety is herein incorporated by reference.

### SEQUENCE LISTING

A Sequence Listing, incorporated herein by reference, is submitted in electronic form as an ASCII text file, created Mar. 24, 2022, of size 23.5 KB, and named “8X56472.TXT”.

### BACKGROUND

### 1. Field

The present disclosure relates to a recombinant microorganism having enhanced ability to remove nitric oxide from a sample, a composition including the recombinant microorganism for reducing a concentration of nitric oxide in a sample, and a method of reducing a concentration of nitric oxide in a sample.

### 2. Description of the Related Art

Nitrogen oxide (NOₓ) is an air pollutant emitted during the combustion process of fuels. Nitrogen oxides (NOₓ) include nitric oxide (NO), nitrous oxide (N₂O), N₂O₃, NO₂, N₂O₄, and N₂O₅. Among these nitrogen oxides, nitric oxide and nitrous oxide are the primary contributors to air pollution. Nitrous oxide absorbs and stores heat in the atmosphere together with carbon dioxide (CO₂), methane (CH₄), and freon gas (e.g., chlorofluorocarbons (CFC)), thereby causing the greenhouse effect. Nitrous oxide is one of the six greenhouse gases subject to regulation by the Kyoto Protocol. Nitrous oxide has a global warming potential (GWP) of 310, which has a higher warming effect per unit mass than carbon dioxide and methane. In addition, nitrogen oxide is the leading cause of smog and acid rain. Nitrogen oxide also undergoes chemical reactions in the air resulting in the production of secondary fine particulate matter (dust) and increases concentrations of ground-level ozone, which adversely affects respiratory health.

Nitrogen oxide removal processes are mostly chemical reduction methods, such as selective catalytic reduction (SCR) and selective non-catalytic reduction (SNCR), and techniques such as scrubbing and adsorption are used. Chemical methods have problems such as costs of energy and catalysts required in the entire process, and treatment of secondary waste generated therefrom. Further, in the case of SCR or SNCR, another greenhouse gas, nitrous oxide, may be generated as a result of incomplete reduction in the process of reducing nitric oxide and nitrous oxide. Unlike chemical techniques having such problems, biological processes are environmentally friendly processes that have advantages such as a relatively simple principle, no use of extreme conditions such as high temperature and high pressure, and low secondary waste or waste water generation. In a biological process, a microorganism serving as a biological catalyst is used instead of a chemical catalyst to oxidize or reduce NOₓ or to fix NOₓ as a part of a cell.

However, despite advances, there remains a need for alternative methods of biological denitrification method.

### SUMMARY

Denitrifying microorganisms reduce nitrogen oxide to N₂ through a dissimilatory reductive process. Various denitrifying microorganisms have been reported in previous studies, such as Pseudomonas putida, Pseudomonas denitrificans, Pseudomonas stutzeri, Paracoccus denitrificans, and Klebsiella pneumonia. Biological denitrification methods using alternative microorganisms would be advantageous.

Disclosed is a recombinant microorganism including a genetic modification that increases expression of a gene encoding a ferric enterobactin transporter-associated protein or a gene encoding a TonB-dependent transporter-associated protein, or a genetic modification that decreases expression of a gene encoding a ferric uptake regulator (Fur) protein, wherein the recombinant microorganism removes greater amounts of nitric oxide from a sample comprising nitric oxide than a same microorganism without the genetic modification.

Also disclosed is a composition for reducing a concentration of nitric oxide in a sample, the composition including a recombinant microorganism including a genetic modification that increases expression of a gene encoding a ferric enterobactin transporter-associated protein or a gene encoding a TonB-dependent transporter-associated protein, or a genetic modification that decreases expression of a gene encoding a ferric uptake regulator (Fur) protein, wherein the recombinant microorganism removes greater amounts of nitric oxide from the sample than a same microorganism without the genetic modification.

Disclosed herein also is a method of reducing a concentration of nitric oxide in a sample including nitric oxide, the method including contacting the sample with a recombinant microorganism including a genetic modification that increases expression of a gene encoding a ferric enterobactin transporter-associated protein or a gene encoding a TonB-dependent transporter-associated protein, or a genetic modification that decreases expression of a gene encoding a ferric uptake regulator (Fur) protein, wherein the recombinant microorganism removes greater amounts of nitric oxide from the sample comprising nitric oxide than a same microorganism without the genetic modification.

Additional aspects will be set forth in part in the description which follows and, in part, will be apparent from the description, or may be learned by practice of the presented embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1A is a graph of relative ¹⁵N₂O (percent, %) versus test sample, showing the amount of ¹⁵N₂O generated by culturing Escherichia. coli (E. coli) W3110ΔfepB and W3110ΔfepDGC strains in the presence of Fe(II)EDTA-NO;

FIG. 1B is an image of serum bottles after E. coli W3110ΔfepB and W3110ΔfepDGC strains were cultured in the presence of Fe(II)EDTA-NO;

FIG. 1C is an image of serum bottles after E. coli W3110ΔfepB and W3110ΔfepDGC strains were cultured in the presence of Fe(II)EDTA-NO;

FIG. 2 is a graph of relative ¹⁵N₂O (%) versus test sample, showing the amount of ¹⁵N₂O generated by culturing overexpressing E. coli strains W3110/pIND4-fepB and W3110/plND4-fepDGC, and E. coli into which an empty vector was introduced (negative control), in the presence of Fe(II)EDTA-NO;

FIG. 3 is a graph of relative ¹⁵N₂O (%) versus test sample, showing the amount of ¹⁵N₂O generated by culturing, overexpressing E. coli strains W3110/pIND4-cirA, W3110/pIND4-fiu, W3110/pIND4-fhuE, W3110/pIND4-fecA, W3110/pIND4-btuB, W3110/pIND4-yncD and W3110/pIND4-tonB, and E. coli into which an empty vector was introduced (negative control), in the presence of Fe(II)EDTA-NO;

FIG. 4A is a graph of remaining Fe(II)EDTA-NO (millimolar, mM) versus time (hours, hr), showing the amount of remaining Fe(II)EDTA-NO after culturing fur gene-deleted E. coli W3110Δfur and a wild-type E. coli as a control, in the presence of Fe(II)EDTA-NO;

FIG. 4B is an image of serum bottles after fur gene-deleted E. coli W3110Δfur and a wild-type E. coli (control) were cultured in the presence of Fe(II)EDTA-NO; and

FIG. 4C is a graph of Fe(II)EDTA-NO removal (%) versus sample, showing the amount Fe(II)EDTA-NO removed after fur gene-deleted E. coli W311OΔfur and a wild-type E. coli (control) were cultured in the presence of Fe(II)EDTA-NO.

### DETAILED DESCRIPTION

The terms “increase in expression” or “increased expression” as used herein refers to detectable increase in the expression of a gene. In particular, an increase in expression means that the expression level of a given gene in a genetically modified (genetically engineered) cell is greater than the expression level of the same gene in a comparative cell of the same type (i.e., the original or “wild-type” cell), without the genetic modification. For example, an expression level of a gene in the modified cell may be increased by about 5% or greater, about 10% or greater, about 15% or greater, about 20% or greater, about 30% or greater, about 50% or greater, about 60% or greater, about 70% or greater, or about 100% or greater, as compared with the expression level of the same gene in an unmodified cell, e.g., a wild-type cell. A cell having increased or enhanced expression of a protein or enzyme may be measured using methods known to those of ordinary skill in the art.

The increase in expression of a gene may be achieved by increasing the copy number of the gene. The term “increase in copy number” as it relates to a gene may result by amplification of a gene or the introduction of an additional copy of a gene. An increase in copy number of a gene may be achieved by genetically engineering a cell to introduce an exogenous gene that is not naturally present in the cell. The introduction of the exogenous gene may be mediated by a vehicle, such as a vector, containing the exogenous gene. The introduction of the gene into the cell may be a transient introduction in which the gene is not integrated into the genome of the cell, or alternatively, may result in integration of the gene into the genome. The introduction may be performed, for example, by introducing a vector into the cell, in which the vector includes a polynucleotide encoding a target polypeptide and replicating the vector in the cell; or by integrating the polynucleotide into the genome. The increase in copy number may refer to several polypeptides which together form a complex and exhibit a single activity, or to multiple copies (i.e., more than one) of a same gene encoding at least one polypeptide constituting the complex.

The introduction of the gene into the cell may be performed by any suitable method, such as, for example, transformation, transfection, and electroporation. The gene may be introduced via a vehicle, or may be introduced by itself. The term “vehicle” as used herein refers to a nucleic acid molecule that is capable of delivering other nucleic acids linked thereto. As a nucleic acid sequence mediating introduction of a specific gene, the vehicle as used herein is construed to be interchangeable with a vector, a nucleic acid construct, and a cassette. Examples of the vector include a plasmid, a virus-derived vector, or the like. A plasmid is a circular double-stranded DNA molecule linkable with another DNA. Examples of the vector include a plasmid expression vector and a virus expression vector, e.g., a replication-defective retrovirus, adenovirus, adeno-associated virus, and a combination thereof.

As used herein “decrease in expression” or “decreased expression” refers to a detectable decrease in the expression of a gene. In particular, a decrease in expression means the expression level of given gene in a modified (e.g., genetically engineered) cell is less than expression of the same gene in a comparative cell of the same type (e.g., the original or “wild-type” cell), without the genetic modification. For example, an expression level of the modified cell may be decreased by about 5% or greater, about 10% or greater, about 15% or greater, about 20% or greater, about 30% or greater, about 50% or greater, about 60% or greater, about 70% or greater, or about 100% or greater, as compared with expression level of the same gene in of an unmodified cell, e.g., a wild-type cell. Decreased expression may be measured by using methods known to those of ordinary skill in the art.

The decrease in expression may be caused by deletion or disruption of a gene encoding a protein. As used herein, the “deletion” or “disruption” of a gene refers to a genetic modification that results in no detectable protein activity for the corresponding protein or to decreased protein activity even when the gene is not expressed, or an amount of gene expression as measured by RNA transcripts is reduced or expressed. The deletion or disruption of a gene is construed as including “inactivation” or “attenuation” of the gene. The genetic modification may be made to the coding region of the gene (the portion of the gene that is transcribed and translated into protein) or to the noncoding region (e.g., promoter region, terminator region) of the gene. The genetic modification may include, for example, mutation, substitution, deletion, or insertion of at least one nucleotide in a gene. Deletion or disruption of the gene may be achieved through gene manipulation methods such as homologous recombination, mutagenesis induction, or molecular evolution. When a cell contains a plurality of copies of the same gene or contains two or more different polypeptide homologous genes (paralogs), one or more genes may be deleted or disrupted.

The gene manipulation may be performed by suitable molecular biological methods.

The term “parent cell” refers to an original cell, e.g., a cell of the same type as the genetically engineered cell but without the genetic modification. With regard to a specific genetic modification, the parent cell may be a cell that lacks the specific genetic modification, but is genetically identical in all other respects to the cell with the genetic modification. Thus, the parent cell is a cell that is used as a starting material to produce a genetically engineered cell having an increased or decreased expression level of a gene encoding a given protein (e.g., a protein having about 75% or greater amino acid sequence identity to a fepB). The same comparison applies to different genetic modifications.

The term “gene” as used herein refers to a nucleic acid fragment encoding the information for expressing a specific protein. The gene may include regulatory sequences such as a 5′ non-coding sequence and/or a 3′ non-coding sequence, or may be free of regulatory sequences.

A “polypeptide” is a polymer chain comprised of amino acid residue monomers which are joined together through amide bonds (peptide bonds). In general, a polypeptide may include at least 10, 20, 50, 100, 200, 500, or more amino acid residue monomers.

The term “sequence identity” of a nucleic acid or polypeptide, as used herein, refers to a degree of identity between nucleotides of polynucleotide sequences or amino acids residues of polypeptides sequences, and is obtained after the sequences are aligned so as to obtain a best match in specific comparable regions. The sequence identity is a value obtained by comparison of two sequences in specific comparable regions via optimal alignment of the two sequences, wherein portions of the sequences in the specific comparable regions may be added or deleted relative to reference sequences. A percentage of sequence identity may be calculated by, for example, comparing two optimally aligned sequences in the entire comparable regions, determining the number of locations in which the same amino acids or nucleotides appear to obtain the number of matching locations, dividing the number of matching locations by the total number of locations in the compared regions (that is, the size of a range), and multiplying the result of the division by 100% to obtain the percentage of the sequence identity. The percentage of the sequence identity may be determined using a known sequence comparison program, for example, BLASTn™ (NCBI), BLASTp™(NCBI), CLC Main Workbench (CLC bio), or MegAlign™(DNASTAR Inc), but is not limited thereto.

The term “genetic modification” as used herein includes artificial alteration in a constitution or structure of genetic materials of a cell.

According to an aspect, a recombinant microorganism having an enhanced ability to remove nitric oxide from a sample is provided. The recombinant microorganism may include a genetic modification that increases gene expression of a gene encoding a ferric enterobactin transporter-associated protein or a gene encoding a TonB-dependent transporter-associated protein, or a genetic modification that may decrease expression of a fur gene encoding a ferric uptake regulator (Fur) protein, wherein the recombinant microorganism removes greater amounts of nitric oxide from a sample comprising nitric oxide than a same microorganism without the genetic modification.

The genetic modification may increase expression of a gene encoding a ferric enterobactin transporter-associated protein such as a fepB gene encoding FepB, a fepDGC gene encoding FepD, FepG, and FepC, or a combination thereof.

FepB, or ferric enterobactin-binding periplasmic protein B, is a protein that present in a periplasmic place and is a ferric enterobactin ABC transporter periplasmic-binding protein.

FepC is an ATP-binding protein, and FepD and FepG are transmembrane proteins. The FepD, FepG, and FepC proteins together form a FepDGC complex which serves as a ferric enterobactin ABC transporter. The FepDGC complex may be present in an inner membrane.

In an aspect, the genetic modification may increase expression of a cirA gene encoding CirA, fiu gene encoding Fiu, a fhuE gene encoding FhuE, a fecA gene encoding FecA, a btuB gene encoding BtuB, a yncD gene encoding YncD, a tonB gene encoding TonB, or a combination thereof.

CirA is an iron-catecholate outer membrane transporter. Fiu is a TonB-dependent iron catecholate transporter. FhuE is a ferric coprogen/ferric rhodotorulic acid outer membrane transporter. FecA is a ferric citrate outer membrane transporter. BtuB is a cobalamin outer membrane transporter. YncD is a putative TonB-dependent outer membrane receptor. TonB is a Ton complex subunit TonB.

The genetic modification may increase a copy number of a fepB gene, a fepDGC gene, or a combination thereof, or the genetic modification may increase a copy number of a cirA gene, a fiu gene, a fhuE gene, a fecA gene, a btuB gene, a yncD gene, a tonB gene, or a combination thereof.

The FepB may be a polypeptide having a sequence identity of 75% or greater, for example, 80% or greater, for example, 85% or greater, for example, 90% or greater, for example, 95% or greater, or for example, 98% or greater, with the amino acid sequence of SEQ ID NO: 1. The fepB gene may be a polynucleotide having a sequence identity of 75% or greater, for example, 80% or greater, for example, 85% or greater, for example, 90% or greater, for example, 95% or greater, or for example, 98% or greater, with the nucleotide sequence of SEQ ID NO: 2.

The FepD may be a polypeptide having a sequence identity of 75% or greater, for example, 80% or greater, for example, 85% or greater, for example, 90% or greater, for example, 95% or greater, or for example, 98% or greater, with the amino acid sequence of SEQ ID NO: 3. The fepD gene may be a polynucleotide having a sequence identity of 75% or greater, for example, 80% or greater, for example, 85% or greater, for example, 90% or greater, for example, 95% or greater, or for example, 98% or greater, with the nucleotide sequence of SEQ ID NO: 4.

The FepG may be a polypeptide having a sequence identity of 75% or greater, for example, 80% or greater, for example, 85% or greater, for example, 90% or greater, for example, 95% or greater, or for example, 98% or greater, with the amino acid sequence of SEQ ID NO: 5. The fepG gene may be a polynucleotide having a sequence identity of 75% or greater, for example, 80% or greater, for example, 85% or greater, for example, 90% or greater, for example, 95% or greater, or for example, 98% or greater, with the nucleotide sequence of SEQ ID NO: 6.

The FepC may be a polypeptide having a sequence identity of 75% or greater, for example, 80% or greater, for example, 85% or greater, for example, 90% or greater, for example, 95% or greater, or for example, 98% or greater, with the amino acid sequence of SEQ ID NO: 7. The fepC gene may be a polynucleotide having a sequence identity of 75% or greater, for example, 80% or greater, for example, 85% or greater, for example, 90% or greater, for example, 95% or greater, or for example, 98% or greater, with the nucleotide sequence of SEQ ID NO: 8.

The CirA may be a polypeptide having a sequence identity of 75% or greater, for example, 80% or greater, for example, 85% or greater, for example, 90% or greater, for example, 95% or greater, or for example, 98% or greater, with the amino acid sequence of SEQ ID NO: 9. The cirA gene may be a polynucleotide having a sequence identity of 75% or greater, for example, 80% or greater, for example, 85% or greater, for example, 90% or greater, for example, 95% or greater, or for example, 98% or greater, with the nucleotide sequence of SEQ ID NO: 10.

The Fiu may be a polypeptide having a sequence identity of 75% or greater, for example, 80% or greater, for example, 85% or greater, for example, 90% or greater, for example, 95% or greater, or for example, 98% or greater, with the amino acid sequence of SEQ ID NO: 11. The fiu gene may be a polynucleotide having a sequence identity of 75% or greater, for example, 80% or greater, for example, 85% or greater, for example, 90% or greater, for example, 95% or greater, or for example, 98% or greater, with the nucleotide sequence of SEQ ID NO: 12.

The FhuE may be a polypeptide having a sequence identity of 75% or greater, for example, 80% or greater, for example, 85% or greater, for example, 90% or greater, for example, 95% or greater, or for example, 98% or greater, with the amino acid sequence of SEQ ID NO: 13. The fhuE gene may be a polynucleotide having a sequence identity of 75% or greater, for example, 80% or greater, for example, 85% or greater, for example, 90% or greater, for example, 95% or greater, or for example, 98% or greater, with the nucleotide sequence of SEQ ID NO: 14.

The FecA may be a polypeptide having a sequence identity of 75% or greater, for example, 80% or greater, for example, 85% or greater, for example, 90% or greater, for example, 95% or greater, or for example, 98 % or greater, with the amino acid sequence of SEQ ID NO: 15. The fecA gene may be a polynucleotide having a sequence identity of 75% or greater, for example, 80% or greater, for example, 85% or greater, for example, 90% or greater, for example, 95% or greater, or for example, 98% or greater, with the nucleotide sequence of SEQ ID NO: 16.

The BtuB may be a polypeptide having a sequence identity of 75 % or greater, for example, 80 % or greater, for example, 85 % or greater, for example, 90 % or greater, for example, 95 % or greater, or for example, 98 % or greater, with the amino acid sequence of SEQ ID NO: 17. The btuB gene may be a polynucleotide having a sequence identity of 75 % or greater, for example, 80 % or greater, for example, 85 % or greater, for example, 90 % or greater, for example, 95 % or greater, or for example, 98 % or greater, with the nucleotide sequence of SEQ ID NO: 18.

The YncD may be a polypeptide having a sequence identity of 75% or greater, for example, 80% or greater, for example, 85% or greater, for example, 90% or greater, for example, 95% or greater, or for example, 98% or greater, with the amino acid sequence of SEQ ID NO: 19. The yncD gene may be a polynucleotide having a sequence identity of 75% or greater, for example, 80% or greater, for example, 85% or greater, for example, 90% or greater, for example, 95% or greater, or for example, 98% or greater, with the nucleotide sequence of SEQ ID NO: 20.

The TonB may be a polypeptide having a sequence identity of 75% or greater, for example, 80% or greater, for example, 85% or greater, for example, 90% or greater, for example, 95% or greater, or for example, 98% or greater, with the amino acid sequence of SEQ ID NO: 21. The tonB gene may be a polynucleotide having a sequence identity of 75% or greater, for example, 80% or greater, for example, 85% or greater, for example, 90% or greater, for example, 95% or greater, or for example, 98% or greater, with the nucleotide sequence of SEQ ID NO: 22.

The Fur may be a ferric uptake regulator, a transcription dual regulator, or the Fur may serve to repress iron acquisition genes when iron is sufficient. The genetic modification may be disruption or deletion of fur gene.

The Fur may be a polypeptide having a sequence identity of 75% or greater, for example, 80% or greater, for example, 85% or greater, for example, 90% or greater, for example, 95% or greater, or for example, 98% or greater, with the amino acid sequence of SEQ ID NO: 23. The fur gene may be a polynucleotide having a sequence identity of 75% or greater, for example, 80% or greater, for example, 85% or greater, for example, 90% or greater, for example, 95% or greater, or for example, 98% or greater, with the nucleotide sequence of SEQ ID NO: 24.

The recombinant microorganism may have ability to reduce nitrogen oxide before or after the genetic modification. The ability may be granted by the recombination. The recombinant microorganism may belong to Pseudomonas genus, Paracoccus genus, or Escherichia genus. The Pseudomonas genus microorganism may be Pseudomonas stutzeri or Pseudomonas aeruginosa. The Paracoccus genus microorganism may be Paracoccus versutus. The Escherichia genus microorganism may be Escherichia coli.

The nitric oxide may be in the form of Fe(ll)(L)-NO. The Fe(ll)(L)-NO is a chelating complex composed of chelating agent L, Fe²⁺, and NO. The chelating agent L may be, for example, ethylene diamine, diethylene triamine, triethylene tetraamine, hexamethylene tetraamine, N-(2-hydroxyethyl)ethylenediamine-triacetic acid (HEDTA), ethylenediamine-tetraacetic acid (EDTA), iminodiacetic acid, nitrilo-triacetic acid (NTA), or diethylene triamine pentaacetic acid (DTPA). Thus, the Fe(ll)(L)-NO may be in a changed form in which nitrogen oxide such as N₂O, NO, N₂O₃, NO₂, N₂O₄, and N₂O₅ is soluble in an aqueous solution. The formation of Fe(ll)(L)-NO may be performed by contacting an aqueous solution containing Fe(ll)(L) with the nitrogen oxide. The contacting may include mixing an aqueous medium with a liquid sample including nitrogen oxide or contacting an aqueous medium with a gaseous sample (e.g., air) including nitrogen oxide. However, the recombinant microorganism is not necessarily construed as being limited to these specific mechanisms in reducing a concentration of nitric oxide in a sample.

In the recombinant microorganism, a genetic modification of a fepB gene encoding FepB, a fepDGC gene encoding FepD, FepG, and FepC, a cirA gene encoding CirA, fiu gene encoding Fiu, a fhuE gene encoding FhuE, a fecA gene encoding FecA, a btuB gene encoding BtuB, a yncD gene encoding YncD, a tonB gene encoding TonB, or a combination thereof may be introduced to the microorganism via a vector. The vector may be present outside a chromosome (i.e., is not integrated into a chromosome of the microorganism).

The recombinant microorganism may reduce a concentration of nitrogen oxide, such as nitric oxide, in a sample containing the nitrogen oxide. The reducing may include converting Fe(ll)(L)-NO to nitrous oxide (N₂O) by a denitrifying enzyme such as nitric oxide reductase or by converting nitrous oxide (N₂O) to N₂ by a denitrifying enzyme such as the nitrous oxide reductase. The sample may be in the form of a liquid or a gas. The sample may be industrial waste water or waste gas. The sample may be any material that includes a nitrogen oxide (e.g., nitric oxide). The nitrogen oxide may include N₂O, NO, N₂O₃, NO₂, N₂O₄, N₂O₅, or a combination thereof. The nitric oxide in the present specification may be NO or Fe(ll)(L)-NO.

According to another aspect, a composition for reducing a concentration of nitric oxide in a sample includes a recombinant microorganism having an enhanced ability to remove nitric oxide from a sample. In an aspect, the composition for reducing concentration of nitric oxide in a sample includes a recombinant microorganism including a genetic modification that increases expression of a gene encoding a ferric enterobactin transporter-associated protein or a gene encoding a TonB-dependent transporter-associated protein, or a genetic modification that decreases expression of a gene encoding a ferric uptake regulator (Fur) protein, wherein the recombinant microorganism removes greater amounts of nitric oxide from the sample than a same microorganism without the genetic modification.

With regard to the composition, the recombinant microorganism, the sample, and the nitric oxide may be the same as described above.

With regard to the composition, the term “reducing” includes decreasing a concentration of nitric oxide present in the sample by any amount, and includes but is not limited to the complete removal of nitric oxide from the sample. The sample may be a gas or a liquid. The composition may further include a material that increases solubility of the nitric oxide for a medium or a culture medium. The nitric oxide may be NO or Fe(ll)(L)-NO.

The composition may reduce a concentration of nitric oxide in the sample by contacting the composition with the sample. The contacting may be performed in a liquid phase. The contacting may be performed, for example, by contacting a culture medium in which the recombinant microorganism is being cultured with the sample containing the nitric oxide. The contacting may be performed under conditions in which the recombinant microorganism is allowed to proliferate. The contacting may be performed in a sealed container. The contacting may be performed under anaerobic conditions. The contacting may include culturing or incubating the recombinant microorganism in the presence of the sample including the nitric oxide. The contacting may include culturing the recombinant microorganism in a sealed container under conditions in which the recombinant microorganism is allowed to proliferate. The medium may be a chemically defined medium. The term “chemically defined medium” as used herein refers to a medium of which the chemical composition is known. The chemically defined medium may not include a complex component such as serum or a hydrolysate. The liquid medium may include LB medium, M9 medium, phosphate buffer, Tris buffer, or a combination thereof. The medium may include Mg²⁺ ions in a range of about 0.1 millimolar (mM) to about 7.5 mM, about 0.5 mM to about 7.5 mM, about 0.5 mM to about 5.0 mM, about 0.5 mM to about 2.5 mM, about 0.5 mM to about 1.5 mM, or about 1.0 mM to about 2.5 mM.

According to another aspect, a method of reducing a concentration of nitric oxide in a sample may include contacting a recombinant microorganism having an enhanced ability to remove nitric oxide with a samples including the nitric oxide. In an aspect, a method of reducing a concentration of nitric oxide in a sample comprising nitric oxide comprises contacting the sample containing nitric oxide with a recombinant microorganism including a genetic modification that increases expression of a gene encoding a ferric enterobactin transporter-associated protein or a gene encoding a TonB-dependent transporter-associated protein, or a genetic modification that decreases expression of a gene encoding a ferric uptake regulator (Fur) protein, wherein the recombinant microorganism removes greater amounts of nitric oxide from the sample comprising nitric oxide than a same microorganism without the genetic modification.

With regard to the method, the recombinant microorganism and the sample including nitric oxide may be the same as those described above.

With regard to the method, the contacting may be performed with the recombinant microorganism in a liquid phase. The contacting may be performed, for example, by contacting a culture medium in which the recombinant microorganism is being cultured with the sample including the nitric oxide. The culturing may be performed under conditions in which the recombinant microorganism may be allowed to proliferate. The contacting may be performed in a sealed container. The contacting may be performed under anaerobic conditions. The medium may be a chemically defined medium. The chemically defined medium may not include a complex component such as serum or hydrolysate. The liquid medium may include LB medium, M9 medium, phosphate buffer, Tris buffer, or a combination thereof. The medium may include Mg²⁺ ions in a range of about 0.1 mM to about 7.5 mM, about 0.5 mM to about 7.5 mM, about 0.5 mM to about 5.0 mM, about 0.5 mM to about 2.5 mM, about 0.5 mM to about 1.5 mM, or about 1.0 mM to about 2.5 mM.

The contacting may be performed during an exponential phase or a stationary phase of growth of the recombinant microorganism. The culturing may be performed under anaerobic conditions. The contacting may be performed in a sealed container under conditions in which the recombinant microorganism may survive or be viable. The conditions in which the recombinant microorganism may survive or be viable may be conditions in which the recombinant microorganism is allowed to proliferate.

With regard to the method, the sample may be in a liquid or gas phase. The sample may be industrial waste water or a waste gas. The sample may be passively or actively contacted with the culture of the recombinant microorganism. The sample may be, for example, sparged into the culture of the recombinant microorganism. That is, the sample may be sparged into a medium or a culture medium. The sparging may include sparging of the sample from the bottom to the top of the medium or from the top to the bottom of the culture medium. The sparging may include injecting of droplets of the sample.

With regard to the method, the contacting may be performed in a batch or continuous manner. The contacting may include, for example, contacting a previously reduced sample (i.e., a sample previously contacted with the recombinant microorganism) with fresh recombinant microorganism. The contacting of the previously reduced sample with the fresh recombinant microorganism may be performed two times or greater, for example, two times, three times, five times, or ten times or greater. The contacting may be continued or repeated until the concentration of nitric oxide in the sample reaches a desired reduced concentration.

According to an aspect, a method of preparing a recombinant microorganism having an enhanced ability to remove nitric oxide from a sample includes introducing a genetic modification which increases expression of a gene encoding a ferric enterobactin transporter-associated protein or a gene encoding a TonB-dependent transporter-associated protein, or a genetic modification that decreases expression of a gene encoding a gene encoding a ferric uptake regulator (Fur) protein. The method may include introducing the genes into the microorganism. The introduction of the genes may be mediated by a vehicle (e.g., a vector such as a plasmid) including the genes.

According to an aspect of the present disclosure, a recombinant microorganism may be used to remove nitric oxide from a sample including the nitric oxide.

According to another aspect, a recombinant microorganism may be used to reduce a concentration of nitric oxide in a sample including the nitric oxide.

According to still another aspect, the disclosed methods may effectively reduce a concentration of nitric oxide in a sample.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects.

The terminology used herein is for the purpose of describing particular embodiments only and is not intended to be limiting. “At least one” is not to be construed as limiting “a” or “an.” As used herein, “a,” “an,” “the,” and “at least one” do not denote a limitation of quantity, and are intended to cover both the singular and plural, unless the context clearly indicates otherwise. For example, “an element” has the same meaning as “at least one element,” unless the context clearly indicates otherwise. “Or” means “and/or.” As used herein, the term “and/or” includes any and all combinations of one or more of the associated listed items. As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ± 30%, 20%, 10% or 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Hereinafter, the inventive concept of the present disclosure will be described in greater detail with reference to Examples. However, these Examples are for illustrative purposes only, and the scope of the present disclosure is not intended to be limited by these Examples.

### EXAMPLES

### Example 1: Preparation of Escherichia Genus Microorganisms in Which Fepb and Fepdgc Genes Are Deleted or Overexpressed

In E. coli of the Example, the fepB and fepDGC genes are each deleted or overexpressed. The ability to remove Fe(II)EDTA-NO from samples using each of the recombinant strains was identified.

### 1. Preparation of Strains in Which Fepb and FepDGC Genes Are Deleted or Overexpressed

In E. coli W3110 strain, deletion of the fepB and fepDGC genes was performed according to a one-step inactivation method described by Datsenko et al (KA Datsenko and BL Wanner, Proc Natl Acad Sci USA. 2000 Jun 6; 97(12):6640-5).

For deletion of the fepB and fepDGC genes, PCR was performed using pKD3 vector as a template and the oligonucleotides of SEQ ID NOs: 25 and 26; and SEQ ID NOs: 29 and 30, were used as primers. The resulting DNA fragments were electroporated into competent cells of the E. coli W3110 strain, expressing λ-red recombinase, to prepare mutant strains in which the fepB and fepDGC genes were each completely deleted. To confirm deletion of the fepB and fepDGC genes, colony PCR was performed using SEQ ID NOs: 27 and 28, and SEQ ID NOs: 31 and 32, as primers. As a result, the W311OΔfepB strain in which fepB gene was deleted and the W3110ΔfepDGC strain in which fepDGC gene was deleted were obtained.

In addition, for deletion of membrane protein genes fhuCDB, fecBCDE, btuF, and btuCED, which are related to iron uptake in E. coli, PCR was performed using pKD3 vector as a template and oligonucleotides of SEQ ID NOs: 33 and 34; and SEQ ID NOs: 37 and 38; SEQ ID NOs: 41 and 42; and SEQ ID NOs: 45 and 46, were used as primers. The resulting DNA fragments were electroporated into competent cells of W3110 strain expressing λ-red recombinase to prepare mutant strains in which genes fhuCDB, fecBCDE, btuF, and btuCED were individually deleted. To confirm deletion of the fhuCDB, fecBCDE, btuF, and btuCED genes, colony PCR was performed by using SEQ ID NOs: 35 and 36; and SEQ ID NOs: 39 and 40; SEQ ID NOs: 43 and 44; and SEQ ID NOs: 47 and 48 as primers. As a result, the E. coli W3110ΔfhuCDB strain in which fhuCDB gene was deleted, the E. coli W3110ΔfecBCDE strain in which fhuBCED gene was deleted, the E. coli W3110ΔbtuF strain in which btuF gene was deleted, and the E. coli W3110ΔbtuCED strain in which btuCED gene was deleted were obtained.

Next, recombinant strains were prepared in which E. coli-derived fepB and fepDGC genes were overexpressed in E. coli W3110. In detail, E. coli W3110 was cultured in a medium, and E. coli genomic DNA (gDNA) was separated and extracted from the culture. PCR was then performed using the gDNA as a template and oligonucleotides of SEQ ID NOs: 53 and 54; and SEQ ID NOs: 55 and 56, as primer sets, to amplify E. coli fepB and fepDGC genes having the nucleotide sequences of SEQ ID NOs: 2, 4, 6, and 8. By using vector plND4 (AC Ind et al. Appl Environ Microbiol. 2009 Oct; 75(20): 6613-5) as a template, and oligonucleotides of SEQ ID NOs: 77 and 78; and SEQ ID NOs: 75 and 76 as primer sets, PCR amplification was performed to obtain vector fragments. The fepB and fepDGC genes were connected to vector plND4 by using InFusion® Cloning Kit (Clontech Laboratories, Inc.) to obtain fepB and fepDGC gene-overexpressed vectors pIND4-fepB and pIND4-fepDGC. The expression of the fepB and fepDGC genes were induced by isopropyl β-D-1-thiogalactopyranoside (IPTG).

By introducing the fepB and fepDGC overexpressed vectors by electroporation (Sambrook, J & Russell, D.W., New York: Cold Spring Harbor Laboratory Press, 2001) into E. coli W3110 cell, Fe(II)EDTA-NO-reducing ability enhanced strains W3110/plND4-fepB and W3110/plND4-fepDGC were obtained. The transformed strains were selected and obtained using an LB plate medium including kanamycin (50 µg/mL).

### 2. Identification of Ability to Remove Fe(II)EDTA-NO of Recombinant E. Coli

The ability of each of the gene deletion strains prepared in Section (1), i.e., E. coli W3110ΔfepB and W3110ΔfepDGC strains, and control strains, i.e., W3110ΔfhuCDB, W3110ΔfecBCDE, W3110ΔbtuF, and W3110ΔbtuCED, to remove Fe(II)EDTA-NO from a sample, was evaluated.

In addition, the ability to remove Fe(II)EDTA-NO from a sample by each of strains W3110/pIND4-fepB and W3110/pIND4-fepDGC, which are gene-overexpressed strains, and E. coli transformed with an empty vector as a control, was also tested.

First, the recombinant E. coli was cultured in an LB medium in an Erlenmeyer flask at a temperature of 37° C. until OD₆₀₀ reached 0.6. Then, 0.1 mM IPTG was added thereto, followed by stirring and culturing at a temperature of 30° C. at 140 rpm overnight to induce gene expression. Then, the cells were collected and used in the Fe(II)EDTA-NO removal reaction thereafter.

The recombinant E. coli cells were added to M9 medium (pH 7.0) containing 5 grams per liter (g/L) of glucose and 5 mM Fe(ll)EDTA-¹⁵NO in a serum bottle, and at a concentration to provide an OD₆₀₀ of 1, to prepare 30 mL of a culture mixture. The culture mixture was added to a 60 mL-serum bottle, followed by stirring and culturing at a temperature of 30° C. at 140 rpm. The serum bottle was kept in an anaerobic chamber to maintain the culture under anaerobic conditions. A control was prepared in the same manner, except that E. coli including an empty vector was used.

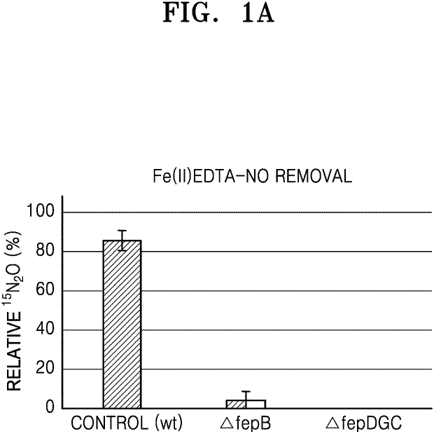

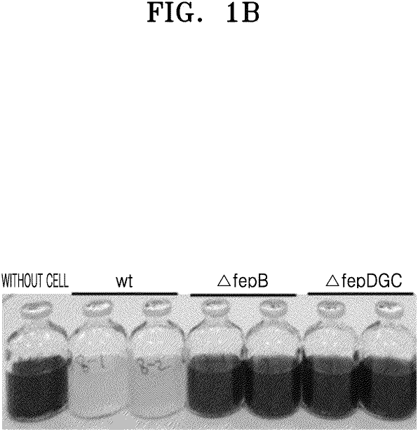

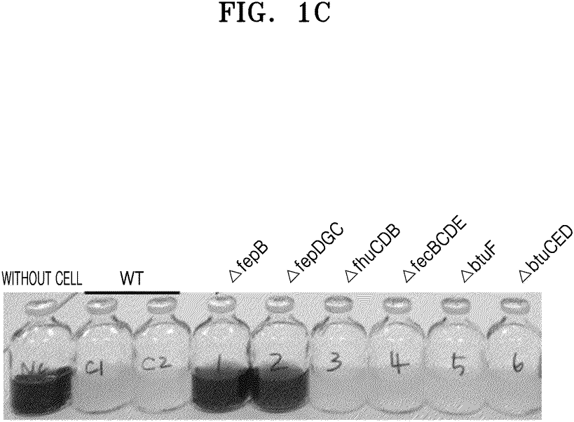

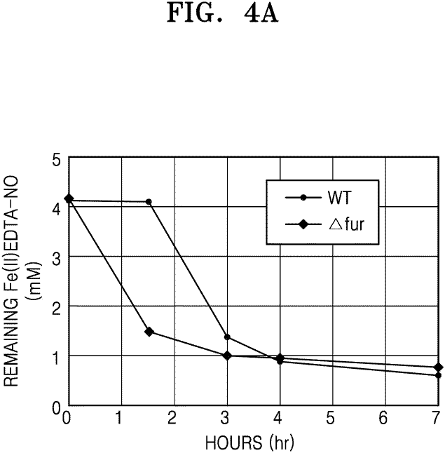

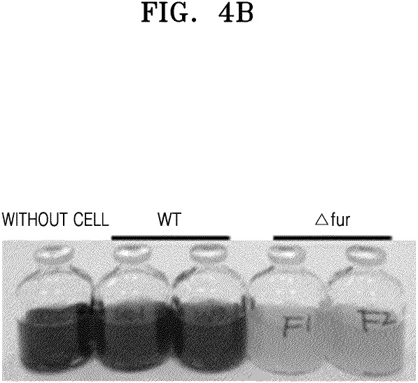

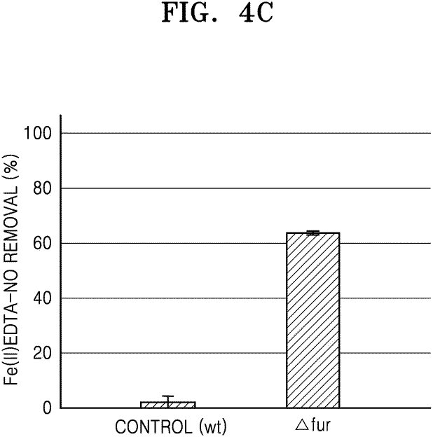

To measure the Fe(ll)EDTA-¹⁵NO concentration, the absorbance A₄₂₀ of a supernatant, obtained by collecting and centrifuging the culture in a serum bottle, was measured to analyze the amount of Fe(ll)EDTA-¹⁵NO remaining in the culture. In addition, the amount of ¹⁵N₂O generated from the reduction of Fe(ll)EDTA-¹⁵NO was analyzed by GC-MS by sampling the gas in the headspace of the reaction serum bottle. The supernatant has a dark brown color at the initial stage when the Fe(II)EDTA-NO complex concentration is high. As the Fe(II)EDTA-NO concentration decreases as a result of reduction to N₂O, the color of the reaction solution gradually transitions from pale to brown, yellow, and then white. Accordingly, as shown in FIGS. 1B, 1C and 4B, the degree of color change of the reaction solution according to the decrease in Fe(II)EDTA-NO concentration may be observed with the naked eye.

FIG. 1A is a graph showing the amount of ¹⁵N₂O generated by culturing E. coli W3110ΔfepB and W3110ΔfepDGC strains in the presence of Fe(II)EDTA-NO. In FIG. 1A, “relative ¹⁵N₂O (%)” represents the ratio of ¹⁵N₂O to the saturated N₂O value obtained in the experimental set. The control was wild-type E. coli.

FIG. 1B is an image of serum bottles after E. coli W3110ΔfepB and W3110ΔfepDGC strains were cultured in the presence of Fe(II)EDTA-NO. The positive control was wild-type E. coli, and the negative control was no addition of a bacterial cell.

FIG. 1C is an image of serum bottles after E. coli W3110ΔfepB and W3110ΔfepDGC strains were cultured in the presence of Fe(II)EDTA-NO. The positive control was with the addition of wild-type E. coli, and the negative control was no bacterial cells, and the comparative (Example) groups were the recombinant E. coli strains W3110ΔfhuCDB, W3110ΔfecBCDE, W3110ΔbtuF, and W3110ΔbtuCED.

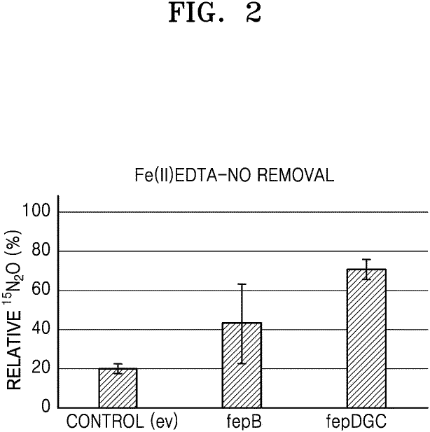

FIG. 2 is a graph showing the amount of ¹⁵N₂O generated by culturing the recombinant E. coli strains W3110/pIND4-fepB and W3110/plND4-fepDGC, and E. coli into which an empty vector was introduced as a control, in the presence of Fe(II)EDTA-NO. In FIG. 2, “relative ¹⁵N₂O (%)” represents the ratio of ¹⁵N₂O to the saturated N₂O value obtained in the experimental set.

As shown in FIGS. 1A and 1B, when the fepB gene and the fepDGC gene are deleted, the amount of ¹⁵N₂O generated from the sample was different (less than) the amount generated from the control.

In addition, as shown in FIG. 1C, when the fepB gene and the fepDGC gene are deleted, the amount of ¹⁵N₂O generated was significantly different (less than), the amount of ¹⁵N₂O generated by E. coli in which fhuCDB, fecBCDE, btuF, or btuCED genes are deleted.

As shown in FIG. 2, when a fepB gene and a fepDGC gene are introduced, the amount of ¹⁵N₂O generated was significant increased, as compared with the control (strain transformed with an empty vector).

### Example 2: Preparation of Strains Overexpressing cirA, Fiu, FhuE, FecA, BtuB, YncD, or TonB Genes in Escherichia Genus Microorganism

The recombinant strains of E. coli W3110, in which a cirA gene, a fiu gene, a fhuE gene, a fecA gene, a btuB gene, a yncD gene, or a tonB gene were overexpressed, were prepared.

In detail, E. coli strain W3110 was cultured in a medium, and E. coli gDNA was separated and extracted from the culture. PCR was performed using the gDNA as a template and oligonucleotides of SEQ ID NOs: 61 and 62; SEQ ID NOs: 63 and 64; SEQ ID NOs: 65 and 66; SEQ ID NOs: 67 and 68; SEQ ID NOs: 69 and 70; SEQ ID NOs: 71 and 72; and SEQ ID NOs: 73 and 74 were used as primer sets to amplify E. coli cirA gene, fiu gene, fhuE gene, fecA gene, btuB gene, yncD gene, and tonB gene respectively. The cirA gene, fiu gene, fhuE gene, fecA gene, btuB gene, yncD gene, and tonB genes have the nucleotide sequences of SEQ ID NOs: 10, 12, 14, 16, 18, 20, and 22, respectively. By using vector plND4 (AC Ind et al. Appl Environ Microbiol. 2009 Oct; 75(20): 6613-5) as a template and oligonucleotides of SEQ ID NOs: 75 and 76 (for insertion of cirA and yncD genes); and SEQ ID NOs: 77 and 78 (for insertion of fiu, fhuE, fecA, btuB, and tonB genes) as primer sets, PCR amplification was performed to obtain vector fragments. The cirA gene, fiu gene, fhuE gene, fecA gene, btuB gene, yncD gene, or tonB genes were inserted into vector plND4 using InFusion® Cloning Kit (Clontech Laboratories, Inc.) to obtain the cirA gene, fiu gene, fhuE gene, fecA gene, btuB gene, yncD gene, and tonB gene-overexpressed vectors pIND4-cirA, pIND4-fiu, pIND4-fhuE, pIND4-fecA, pIND4-btuB, pIND4-yncD, and IND4-tonB, respectively. The expression of each of the genes was induced by IPTG.

The cirA gene, fiu gene, fhuE gene, fecA gene, btuB gene, yncD gene, and tonB gene-overexpressed vectors were introduced into E. coli W3110 by electroporation (Sambrook, J & Russell, D.W., New York: Cold Spring Harbor Laboratory Press, 2001) to prepare recombinant strains W3110/pIND4-cirA, W3110/pIND4-fiu, W3110/pIND4-fhuE, W3110/pIND4-fecA, W3110/pIND4-btuB, W3110/pIND4-yncD, and W3110/pIND4-tonB with enhanced Fe(II)EDTA-NO-reducing ability. The transformed strains were selected and obtained on a LB plate medium including kanamycin (50 µg/mL). Consequently, the recombinant E. coli strains including the overexpressed genes were prepared and were cultured in the presence of Fe(ll)EDTA-¹⁵NO to analyze the amount of ¹⁵N₂O generated.

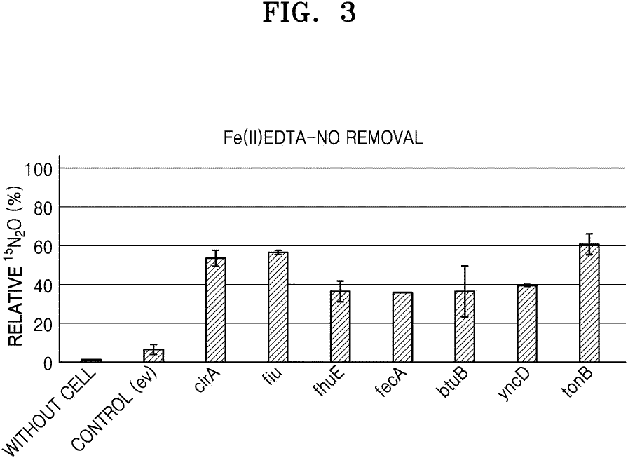

FIG. 3 is a graph showing the amount of ¹⁵N₂O generated by culturing, in the presence of Fe(II)EDTA-NO, the overexpressing E. coli recombinant strains W3110/pIND4-cirA, W3110/pIND4-fiu, W3110/pIND4-fhuE, W3110/pIND4-fecA, W3110/pIND4-btuB, W3110/pIND4-yncD, and W3110/pIND4-tonB, and E. coli into which an empty vector was introduced as a control. In FIG. 3, “relative ¹⁵N₂O (%)” represents the ratio of ¹⁵N₂O to the saturated N₂O value obtained in the experimental set.

### Example 3: Preparation of Recombinant Escherichia Genus Microorganism With Fur Gene Deletion

A one-step inactivation method (KA Datsenko and BL Wanner, Proc Natl Acad Sci U S A. 2000 Jun 6; 97(12):6640-5) was used to delete a fur gene in E. coli W3110 strain.

For deletion of a fur gene, PCR was performed using pKD3 vector as a template and oligonucleotides of SEQ ID NOs: 49 and 50 as primers. The resulting DNA fragment was electroporated to a competent cell of W3110 strain in which A-red recombinase was expressed. To prepare mutant strains in which a fur gene was deleted. To confirm deletion of the fur gene, colony PCR was performed by using SEQ ID NOs: 51 and 52 as primers. Consequently, the W3110 Δfur strain, in which a fur gene was deleted, was obtained. This strain was cultured in the presence of Fe(ll)EDTA-¹⁵NO to analyze the amount of ¹⁵N₂O generated.

FIG. 4A is a graph showing the amount of remaining Fe(II)EDTA-NO in the sample over time. The recombinant fur gene-deleted E. coli W311OΔfur and a wild-type E. coli as a control were cultured in the presence of Fe(II)EDTA-NO and a portion of the sample was withdrawn at various times and the level of Fe(II)EDTA-NO remaining was measured. In FIG. 4A, the remaining Fe(II)EDTA-NO is a quantitative value.

FIG. 4B is an image of serum bottles after the recombinant E. coli W311OΔfur strain and a wild-type E. coli as a control were cultured in the presence of Fe(II)EDTA-NO. The positive control was cultured with wild-type E. coli, and the negative control was cultured without the addition of a cell.

FIG. 4C is a graph showing the percentage of Fe(II)EDTA-NO removed from the sample when the recombinant E. coli W3110Δfur strain and a wild-type E. coli were cultured in the presence of Fe(II)EDTA-NO.

As shown in FIGS. 4A to 4C, when a fur gene-deleted E. coli ( E. coli W3110Δfur) was cultured in the presence of Fe(II)EDTA-NO, a rate of Fe(II)EDTA-NO removal was significantly greater than the wild type E. coli.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A recombinant microorganism comprising a genetic modification that increases expression of a gene encoding a ferric enterobactin transporter-associated protein or a gene encoding a TonB-dependent transporter-associated protein, or a genetic modification that decreases expression of a gene encoding a ferric uptake regulator (Fur) protein, wherein the recombinant microorganism removes greater amounts of nitric oxide from a sample comprising nitric oxide than a same microorganism without the genetic modification.

2. The recombinant microorganism of claim 1, wherein the genetic modification increases expression of a fepB gene encoding FepB, a fepDGC gene encoding FepD, FepG and FepC, or a combination thereof, or the genetic modification increases expression of a cirA gene encoding CirA, a fiu gene encoding Fiu, a fhuE gene encoding FhuE, a fecA gene encoding FecA, a btuB gene encoding BtuB, a yncD gene encoding YncD, a tonB gene encoding TonB, or a combination thereof.

3. The recombinant microorganism of claim 2, wherein the genetic modification increases a copy number of the fepB gene, the fepDGC gene, or a combination thereof, or the genetic modification increases a copy number of the cirA gene, the fiu gene, the fhuE gene, the fecA gene, the btuB gene, the yncD gene, the tonB gene, or a combination thereof.

5. The recombinant microorganism of claim 2, wherein
the FepB is a polypeptide having a sequence identity of 75% or greater with the amino acid sequence of SEQ ID NO: 1,
the FepD is a polypeptide having a sequence identity of 75% or greater with the amino acid sequence of SEQ ID NO: 3,
the FepG is a polypeptide having a sequence identity of 75% or greater with the amino acid sequence of SEQ ID NO: 5,
the FepC is a polypeptide having a sequence identity of 75% or greater with the amino acid sequence of SEQ ID NO: 7,
the CirA is a polypeptide having a sequence identity of 75% or greater with the amino acid sequence of SEQ ID NO: 9,
the Fiu is a polypeptide having a sequence identity of 75% or greater with the amino acid sequence of SEQ ID NO: 11,
the FhuE is a polypeptide having a sequence identity of 75% or greater with the amino acid sequence of SEQ ID NO: 13,
the FecA is a polypeptide having a sequence identity of 75% or greater with the amino acid sequence of SEQ ID NO: 15,
the BtuB is a polypeptide having a sequence identity of 75% or greater with the amino acid sequence of SEQ ID NO: 17,
the YncD is a polypeptide having a sequence identity of 75% or greater with the amino acid sequence of SEQ ID NO: 19, and
the TonB is a polypeptide having a sequence identity of 75% or greater with the amino acid sequence of SEQ ID NO: 21.

5. The recombinant microorganism of claim 1, wherein the genetic modification that decreases expression of the gene encoding the ferric uptake Fur protein is a disruption in the expression of the fur gene or a complete or partial deletion of the fur gene.

6. The recombinant microorganism of claim 1, wherein the Fur protein is a polypeptide having 75% or greater sequence identity with the amino acid sequence of SEQ ID NO: 23.

7. The recombinant microorganism of claim 1, wherein the recombinant microorganism belongs to the genus Escherichia, the genus Pseudomonas, or the genus Paracoccus.

8. A composition for reducing concentration of nitric oxide in a sample, the composition comprising a recombinant microorganism comprising a genetic modification that increases expression of a gene encoding a ferric enterobactin transporter-associated protein or a gene encoding a TonB-dependent transporter-associated protein, or a genetic modification that decreases expression of a gene encoding a ferric uptake regulator (Fur) protein, wherein the recombinant microorganism removes greater amounts of nitric oxide from the sample than a same microorganism without the genetic modification.

9. The composition of claim 8, wherein the genetic modification increases expression of a fepB gene encoding FepB, a fepDGC gene encoding FepD, FepG and FepC, or a combination thereof, or the genetic modification increases expression of a cirA gene encoding CirA, a fiu gene encoding Fiu, a fhuE gene encoding FhuE, a fecA gene encoding FecA, a btuB gene encoding BtuB, a yncD gene encoding YncD, a tonB gene encoding TonB, or a combination thereof.

10. The composition of claim 9, wherein the genetic modification increases a copy number of the fepB gene, the fepDGC gene, or a combination thereof, or the genetic modification increases a copy number of the cirA gene, the fiu gene, the fhuE gene, the fecA gene, the btuB gene, the yncD gene, the tonB gene, or a combination thereof.

11. The composition of claim 9, wherein
the FepB is a polypeptide having a sequence identity of 75% or greater with the amino acid sequence of SEQ ID NO: 1,
the FepD is a polypeptide having a sequence identity of 75% or greater with the amino acid sequence of SEQ ID NO: 3,
the FepG is a polypeptide having a sequence identity of 75% or greater with the amino acid sequence of SEQ ID NO: 5,
the FepC is a polypeptide having a sequence identity of 75% or greater with the amino acid sequence of SEQ ID NO: 7,
the CirA is a polypeptide having a sequence identity of 75% or greater with the amino acid sequence of SEQ ID NO: 9,
the Fiu is a polypeptide having a sequence identity of 75% or greater with the amino acid sequence of SEQ ID NO: 11,
the FhuE is a polypeptide having a sequence identity of 75% or greater with the amino acid sequence of SEQ ID NO: 13,
the FecA is a polypeptide having a sequence identity of 75% or greater with the amino acid sequence of SEQ ID NO: 15,
the BtuB is a polypeptide having a sequence identity of 75% or greater with the amino acid sequence of SEQ ID NO: 17,
the YncD is a polypeptide having a sequence identity of 75% or greater with the amino acid sequence of SEQ ID NO: 19, and
the TonB is a polypeptide having a sequence identity of 75% or greater with the amino acid sequence of SEQ ID NO: 21.

12. The composition of claim 8, wherein the genetic modification that decreases expression of the gene encoding the ferric uptake Fur protein is a disruption in the expression of a fur gene or a complete or partial deletion of the fur gene.

13. The composition of claim 8, wherein the Fur protein is a polypeptide having a sequence identity of 75% or greater with the amino acid sequence of SEQ ID NO: 23.

14. The composition of claim 8, wherein the recombinant microorganism belongs to the genus Escherichia, the genus Pseudomonas, or the genus Paracoccus.

15. A method of reducing a concentration of nitric oxide in a sample comprising nitric oxide, the method comprising contacting the sample with a recombinant microorganism comprising a genetic modification that increases expression of a gene encoding a ferric enterobactin transporter-associated protein or a gene encoding a TonB-dependent transporter-associated protein, or a genetic modification that decreases expression of a gene encoding a ferric uptake regulator (Fur) protein, wherein the recombinant microorganism removes greater amounts of nitric oxide from the sample comprising nitric oxide than a same microorganism without the genetic modification.

16. The method of claim 15, wherein the genetic modification increases expression of a fepB gene encoding FepB, a fepDGC gene encoding FepD, FepG and FepC, or a combination thereof, or the genetic modification increases expression of a cirA gene encoding CirA, a fiu gene encoding Fiu, a fhuE gene encoding FhuE, a fecA gene encoding FecA, a btuB gene encoding BtuB, a yncD gene encoding YncD, a tonB gene encoding TonB, or a combination thereof.

17. The method of claim 16, wherein the genetic modification increases a copy number of the fepB gene, the fepDGC gene, or a combination thereof, or the genetic modification increases a copy number of the cirA gene, the fiu gene, the fhuE gene, the fecA gene, the btuB gene, the yncD gene, the tonB gene, or a combination thereof.

18. The method of claim 16, wherein
the FepB is a polypeptide having a sequence identity of 75% or greater with the amino acid sequence of SEQ ID NO: 1,
the FepD is a polypeptide having a sequence identity of 75% or greater with the amino acid sequence of SEQ ID NO: 3,
the FepG is a polypeptide having a sequence identity of 75% or greater with the amino acid sequence of SEQ ID NO: 5,
the FepC is a polypeptide having a sequence identity of 75% or greater with the amino acid sequence of SEQ ID NO: 7,
the CirA is a polypeptide having a sequence identity of 75% or greater with the amino acid sequence of SEQ ID NO: 9,
the Fiu is a polypeptide having a sequence identity of 75% or greater with the amino acid sequence of SEQ ID NO: 11,
the FhuE is a polypeptide having a sequence identity of 75% or greater with the amino acid sequence of SEQ ID NO: 13,
the FecA is a polypeptide having a sequence identity of 75% or greater with the amino acid sequence of SEQ ID NO: 15,
the BtuB is a polypeptide having a sequence identity of 75% or greater with the amino acid sequence of SEQ ID NO: 17,
the YncD is a polypeptide having a sequence identity of 75% or greater with the amino acid sequence of SEQ ID NO: 19, and
the TonB is a polypeptide having a sequence identity of 75% or greater with the amino acid sequence of SEQ ID NO: 21.

19. The method of claim 15, wherein the genetic modification that decreases expression of the gene encoding the ferric uptake Fur protein is a disruption in the expression of the fur gene or a complete or partial deletion of the fur gene.

20. The method of claim 15, wherein the Fur is a polypeptide having a sequence identity of 75% or greater with the amino acid sequence of SEQ ID NO: 23.

21. The method of claim 15, wherein the recombinant microorganism belongs to the genus Escherichia, the genus Pseudomonas, or the genus Paracoccus.

22. The method of claim 15, wherein the contacting is performed under anaerobic conditions.

23. The method of claim 15, wherein the contacting comprises culturing or incubating the recombinant microorganism in the presence of the sample comprising the nitric oxide.
